(19) United States
(12) Patent Application Publication
(10) Pub. No.: US 2026/0208183 A1
(43) Pub. Date: Jul. 23, 2026

(54) SOLID-BODY SEPARATOR FOR SEPARATING AT LEAST TWO CONSTITUENTS OF A BODILY LIQUID

(71) Applicant: SARSTEDT AG & CO. KG, Nuembrecht (DE)

(72) Inventor: Mark WEINSTOCK, Helmenzen (DE)

(21) Appl. No.: 19/447,613

(22) Filed: Jan. 13, 2026

Related U.S. Application Data
(63) Continuation of application No. PCT/EP2024/070210, filed on Jul. 17, 2024.

(30) Foreign Application Priority Data
Jul. 19, 2023 (DE) 102023119103.9

(51) Int. Cl. B01L 3/00; A61B 5/15; B04B 5/04

(52) U.S. Cl. CPC B01L 3/50215; B04B 5/0414; A61B 5/150755; B01L 2200/0647; B01L 2200/0689; B01L 2300/123; B01L 2400/0409; B01L 2400/06

(57) ABSTRACT

A solids separator, comprising: an axis; a ballast weight; a float, wherein the solids separator is configured to separate two components of a bodily fluid, wherein a first component of the bodily fluid has a first density and a second component of the body fluid has a second density, and wherein a total density of the solids separator is between the first density and the second density, wherein the ballast weight and the float are movable relative to each other under an influence of a centrifugal force acting upon the solids separator; wherein the solids separator includes an elastically deformable connection element forming a force transmitting connection between the float and the ballast weight, so that a relative movement between the float and the ballast weight from an idle position requires a force to overcome a reset force caused by the connection element.

### RELATED APPLICATIONS

This application is a continuation of international application PCT/EP2024/070210 filed on Jul. 17, 2024 that claims priority from German patent application DE 10 2023 119 103.9, filed on Jul. 19, 2023, both of which are incorporated in their entirety by this reference.

### FIELD OF THE INVENTION

The invention relates to a solids separator, configured to separate at least two components of a bodily fluid. A first component of the bodily fluid has a first density, and a second component of the bodily fluid has a second density. A total density of the solids separator is between the first density and the second density of the components to be separated.

### BACKGROUND OF THE INVENTION

Solids separators are used to separate components of a bodily fluid from one another. Thus, a first component of the bodily fluid has a first density and a second component of the bodily fluid has a second density. The bodily fluid can be blood, in particular, wherein the components to be separated are blood, serum, and blood plasma and the leukocyte film or buffy coat. However, any other bodily fluid can be separated into its components and maintained in a separated state using the solids separator. The solids separator can also be used e.g. for separating urine, saliva, amniotic fluid, and intestinal fluid into their respective components.

The solids separator typically includes a first material and a second material wherein both materials are provided in the solids separator separate from each other.

Typically, a solids separator is used in combination with a centrifuge in order to separate the components of the bodily fluid from one another. The solids separator, together with a bodily fluid, is thus inserted into a sample container, typically a test tube, and centrifuged thereafter. The components separate from each other due to the centrifugal force impacting the bodily fluid and due to the different densities of the two components of the bodily fluid. Since this condition is only maintained as long as the centrifugal force impacts the bodily fluid, the solids separator is necessary. A nominal value of a total density of the solids separator is thus provided between the densities of the two components, so that the solids separator moves between the two components during centrifuge operations. Thus, the solids separator is typically configured so that an exchange of the components during centrifuge operations is facilitated, but prevented when the centrifugal force is removed. This way, the components of the bodily fluid remain separated from each other even after centrifuge operations, meaning, after the centrifugal force is removed.

Solids separators are well known in the art, e.g. from EP 3 778 027 A1. The solids separator described therein includes a ballast weight and a float, connected to each other by an elastically deformable connection element. The float includes a pass-through opening, and the ballast weight includes a trunnion, wherein the pass-through opening forms a valve together with the trunnion. The trunnion is inserted into the pass-through opening in an idle position of the solids separator. The connection element thus overlaps with the ballast weight in an overlap portion. Thus, the ballast weight includes four grooves, wherein the connection element that includes four support bars inserted into the grooves is embedded in the four grooves. The ballast weight moves away from the float under the impact of the centrifugal force since the ballast weight has a higher density than the float. This retracts the trunnion from the pass-through opening, releasing the pass-through opening so that the components can flow through. Thus, the elastically deformable connection element assures that the trunnion of the ballast weight moves back into the pass-through opening after the centrifugal force ceases, so that the components are permanently separated from each other.

A recognized problem of the known solids separator has been that the connection element deforms unevenly under force impact, in particular under an impact of a centrifugal force; therefore, the ballast weight is not inserted into the pass-through opening reliably after the centrifugal force is removed and thus a re-mixing of the previously separated components is not reliably prevented. The uneven deformation of the connection element in the overlap portion is caused, in particular, by a friction of the support bar portions of the connection elements in the grooves.

### BRIEF SUMMARY OF THE INVENTION

Thus, it is an object of the invention to provide a solids separator that overcomes the known problems and that is characterized by a reliable transition of the solids separator into an idle condition.

The invention relates to a solids separator, configured to separate at least two components of a bodily fluid. A first component of the bodily fluid has a first density, and a second component of the bodily fluid has a second density. A total density of the solids separator is between the first density and the second density of the components to be separated. The solids separator includes at least one ballast weight and at least one float. The ballast weight and the float are moveable relative to each other, in particular when a centrifugal force impacts the solids separator.

The solids separator additionally includes a deformable connection element. The connection element provides a force-transferring connection between the float and the ballast weight. This way, a force overcoming a reset force determined by the connection element is required for the relative movement between the float and the ballast weight starting from an idle position of the solids separator. Thus, the connection element overlaps the ballast weight or the float in at least one overlap portion. The overlap portion according to the invention is a portion where the connection element overlaps the ballast weight; this means that the connection element is arranged parallel to and separate from the ballast weight. The connection element can be embedded, in particular, into the ballast weight or the float. Thus, the ballast weight or the float includes a recess or a groove. The connection element can also sit on top of the ballast weight or on top of the float and thus protrude beyond a surface of the ballast weight or float. A combination of both variants is also conceivable.

Improving upon the known method, the object is achieved by a solids separator, comprising: an axis a ballast weight; a float, wherein the solids separator is configured to separate two components of a bodily fluid, wherein a first component of the bodily fluid has a first density and a second component of the body fluid has a second density, and wherein a total density of the solids separator is between the first density and the second density of the two components of the bodily fluid that is to be separated, wherein the ballast weight and the float are movable relative to each other under an influence of a centrifugal force acting upon the solids separator; wherein the solids separator includes an elastically deformable connection element forming a force transmitting connection between the float and the ballast weight, so that a relative movement between the float and the ballast weight from an idle position requires a force to overcome a reset force caused by the connection element, wherein the connection element axially overlaps the ballast weight or the float in an overlap portion, wherein the connection element is connected with the ballast weight or the float in an anchoring location, so that a deformation of the connection element in a section of the overlap portion is prevented during the relative movement between the ballast weight and the float caused by a centrifugal force impacting the solids separator floating in the bodily fluid.

Connecting the connection element with the ballast weight or the float prevents a deformation of the connection element at least in a section of the overlap portion when a relative movement of the ballast weight and the float occurs, in particular, caused by an impact of a centrifugal force upon the solids separator floating in the bodily fluid.

Thus, the connection of the connection element with the ballast weight or the float advantageously assures that the connection element cannot deform in the transition portion. This way, it can be assured that the solids separator is reliably transferrable back into the idle position after the centrifugal force ceases. In particular, unpredictability with respect to a safe transfer of the solids separator into the idle position caused by an uneven deformation of the connection element can be prevented. The deformation of the connection element is thus prevented by a type of connection of the connection element with the ballast weight or the float in a connection location.

Advantageously, the connection element is divided into two portions, a deformation of the connection element is enabled in a first portion in order to enable a relative movement between the float and the ballast weight and simultaneously assure that the solids separator can be moved back into the idle position in particular after the centrifugal force ceases. This is necessary, in particular, when a separation of the components is only facilitated by releasing a pass-through opening in the solids separator, wherein the pass-through opening is opened when the ballast weight and the float move away from each other. The first portion can be provided, in particular, as at least one lobe-shaped section. A second portion of the connection element is not deformed even under an impact of the centrifugal force. The second portion can be configured, in particular, to retain the ballast weight at the float during impact of the centrifugal force. The second portion can be configured, in particular, as at least one support bar. Thus, the two portions of the connection element have the advantageous effect that the ballast weight and the float can move relative to each other, but cannot separate from each other completely while it is being simultaneously assured that the connection element does not deform in the overlap portion, which would cause a detrimental unreliable transition of the solids separator into the idle position.

The configuration of the two portions of the connection element can be implemented by different material thicknesses. The first portion can be provided particularly thin, and the second portion can be provided particularly thick, so that a force required for deforming the second portion is increased over a force required for deforming the first portion. Additionally, a contact portion between the first and the second portion can be configured as small as possible. Advantageously, the contact portion is limited to a size that is required for connecting both portions. The two portions can thus operate mechanically as two connected springs that have different spring constants and that are connected in series. This way, the connection element can be prevented from deforming in the overlap portion. This can be implemented by the second portion acting as a spring, having a higher spring constant than the first portion acting as a spring. Thus, the overlap portion can be advantageously arranged in the second portion of the connection element.

According to an advantageous embodiment of the invention, the ballast weight can be connected with the float by friction locking or by positive form locking. In any case, it has to be prevented that the ballast weight separates from the float in its entirety. Advantageously, the float and the connection element of the solids separator are connected with one another by positive form locking, wherein the connection element overlaps the ballast weight in the overlap portion.

According to an advantageous embodiment, the connection element includes at least one lobe-shaped section. Lobe-shaped, according to the invention, is a shape which has a particularly small thickness compared to its width and height and which is therefore deformable particularly easily. Advantageously, the connection element is made from a synthetic material, in particular a thermal plastic elastomeric material. It is an advantage of a lobe-shaped configuration of the connection element that a connection between the float and the ballast weight can be established that requires very little material. Advantageously, the connection element can be configured and oriented so that the connection element deforms along a broad side or an elevation side as long as the connection element is loaded with a force. Particularly advantageously, the connection element deforms under a reduction of the thickness of the connection element.

According to an advantageous embodiment of the invention, the connection element is essentially made from the same material as the float. In order to have a different thickness, the ballast weight can be made from a different material. Thus, the connection element can be advantageously configured integrally in one piece with the float so that the float and the connection element form a single component, whereas the ballast weight forms a second component.

According to the instant invention, a component is a part of the solids separator. The component does not necessarily have to be configured separately from another component or other elements of the solids separator. The component can, rather, also be configured integrally in one piece with other components and/or elements of the solids separator.

Providing the solids separator as described supra is particularly advantageous for production of the solids separator since only two components have to be fabricated to form the solids separator.

According to an advantageous embodiment of the invention, the connection element is connected with the ballast weight or the float by at least one positively form locking anchoring element, wherein the anchoring element is advantageously configured as a bar or as a trunnion. The connection element functions to facilitate a movement between the float and the ballast weight. Simultaneously, it shall be assured that the ballast weight and the float cannot separate completely and that the ballast weight can be transferred back into an idle position after a force impacting the ballast weight, in particular, a centrifugal force, is removed. Thus, it has proven particularly advantageous to provide form locking between the ballast weight and the float, wherein the form locking is provided by the anchoring element. The ballast element may include the anchoring element configured as a bar, whereas the connection element is integrally provided in one piece with the float, wherein the anchoring element is connected with the connection element by positive form locking, e.g. enveloped by the connection element. This provides a particularly reliable connection between the float and the ballast weight.

According to another advantageous embodiment of the invention, the float and/or the ballast weight includes at least one groove in the overlap portion.

Advantageously, at least one support bar can be associated with the connection element, wherein the at least one support bar envelops the ballast weight at least partially. Thus, the support bar is advantageously formed from the same material as the connection element, and further advantageously from the same material as the float. In a particularly advantageous embodiment, the float, the connection element, and the support bar associated therewith are integrally made from the same first material, whereas the ballast weight is made as a discrete component from a second material that has a thickness that differs from a thickness of the first material. The support bar envelops the ballast weight, so that a complete separation of the ballast weight from the float is prevented even when a force is applied. Advantageously, a plurality of support bars is provided, so that retention safety of the float or the ballast bar is improved.

According to another embodiment of the invention, the connection element includes four lobe-shaped sections and four support bars associated with the lobe-shaped sections, wherein the lobe-shaped sections and the associated support bars are respectively arranged at an angle of 90 degrees relative to each other. When a force impacts the ballast weight or the float, the force can be evenly distributed over the four lobe-shaped sections or support bars. Additionally, material loading is reduced and safety is increased.

Particularly advantageously, the support bars converge in an end portion of the solids separator. Thus, the support bars can form a basket in which the ballast weight or the float can be received. This way, the ballast weight or the float can be retained reliably.

According to an advantageous embodiment of the invention, the float includes a sealing portion, wherein the sealing portion is bonded to the support bar by the connection element. The sealing portion facilitates inserting the solids divider into a sample container in a sealing manner. In particular, the sealing portion can contact the wall of the sample container to establish a seal. This can prevent, in particular, that components re-mix again that have been separated from one another after a centrifugation process. Thus, the sealing portion can be provided in particular integrally in one piece with the connection element and the support bar, whereas the connection element connects the sealing portion with the support bar.

According to another advantageous embodiment of the invention, a cross-section surface of the support bar exceeds a cross-section surface of the lobe-shaped section. The support bar at least partially envelops the float or the ballast weight. Thus, in order to provide the best support possible, the cross-section surface of the support bar shall be sized so that support is provided. The lobe-shaped section of the connection element, however, establishes a force transferring connection between the float and the ballast weight. The connection element primarily has to be elastically deformable in the lobe-shaped section. A cross-sectional surface of the lobe shaped section of the connection element can thus be selected at will as long as the function described supra is performed. A combination with a cross-sectional surface of the support bar that is increased over a cross-sectional surface of the lobe-shaped section has proven particularly advantageous for the reasons stated supra.

According to another advantageous embodiment of the invention, the ballast weight includes a first section and a second section, wherein the first section is advantageously provided semi-spherical and the second section is advantageously provided trunnion-shaped. Advantageously, the trunnion-shaped portion is arranged on a top side of the semi-spherical portion. Further advantageously, the float includes a pass-through opening, wherein the second section of the ballast weight is insertable into the pass-through opening, forming a valve. Put differently, the float and the ballast weight jointly form a valve, in which the trunnion of the ballast weight is insertable into the pass-through opening in order to seal the pass-through opening of the float. The trunnion is inserted into the pass-through opening in the idle position of the solids separator, so that a flow of the bodily fluid through the pass-through opening is prevented. The trunnion is retained in the pass-through opening by the connection element. The ballast weight moves away from the float due to the density of the ballast weight when a force, in particular, a centrifugal force, impacts the solids separator. Thus, the trunnion is moved out of the pass-through opening and unblocks the pass-through opening, so that the bodily fluid, in particular, certain components of the bodily fluid, can flow through the pass-through opening. When the solids separator includes a sealing portion, the exchange of components may only occur through the pass-through opening. The connection element assures that the ballast weight does not separate from the float completely. When the force ceases, the connection element causes the trunnion of the ballast weight to be inserted into the pass-through opening due to an elastic deformability of the connection element, so that separated components of the bodily fluid are permanently separated, this means also after the force has ceased to exist.

According to another advantageous embodiment of the invention, the first section of the ballast weight includes the overlapping portion, wherein the overlapping portion is configured as a groove circumferentially enveloping the first section of the ballast weight at least partially.

According to another advantageous embodiment of the invention, the support bar runs in the groove of the ballast weight. As a consequence, the float that is connected with the connection element, in this case, is connected with the ballast weight as well. In this configuration, it can be provided that the connection element deforms in a portion connecting the float and the ballast weight when a force is applied, whereas a deformation in the overlap portion is prevented. Put differently, the support bars associated with the connection element do not deform, so that the support bars support the ballast weight, wherein a force applied by the support bars to the ballast weight is independent from an external force impacting the solids separator. This advantageously prevents the connection element from deforming unevenly due to a friction of the support bars in the groove which would lead to an incomplete closure of the pass-through opening, which would cause an undesirable remixing of the separated components of the bodily fluid.

According to another advantageous embodiment of the invention, the connection element is deformable by a relative movement between the float and the ballast weight, in particular, caused by a centrifugal force impacting the solids separator, so that a contact between the connection element and the first section of the ballast weight outside of the anchoring location is temporarily disengageable at least in sections. Put differently, the connection element separates from the ballast weight outside of the anchoring location. This way, the connection element is expanded, in particular, in an area of the anchoring location, so that the solids separator is impacted by a force that causes a movement of the ballast weight. The elastically deformable connection element causes the contact between the connection element and the first section of the ballast weight to be reestablished outside of the anchoring location after the force ceases to be applied.

According to an advantageous embodiment the first density is greater than 1.05 g/cm³, the second density is less than 1.03 g/cm³, and the total density is in a range of 1.03 g/cm³ to 1.05 g/cm³. The total density is critical to position the solids separator between the blood plasma and the blood serum.

### BRIEF DESCRIPTION OF THE DRAWINGS

The invention is subsequently described with reference to drawing figures herein.

FIG. 1 illustrates a vertical sectional view through a sample container where a solids separator according to the invention is arranged in the sample container in an idle position;

FIG. 2 illustrates a perspective view of a float of the solids separator according to FIG. 1;

FIG. 3 illustrates a perspective view of a ballast weight of the solids separator according to FIG. 1;

FIG. 4 illustrates another perspective view of the ballast weight according to FIG. 3 in a vertical sectional view;

FIG. 5 illustrates a perspective view of the solids separator according to FIG. 1;

FIG. 6 illustrates a vertical sectional view of the solids separator according to FIG. 1;

FIG. 7 illustrates a vertical sectional view through the solids separator according to FIG. 6, wherein the solids separator is impacted by a centrifugal force;

FIG. 8 illustrates a horizontal sectional view of a sample container, wherein the solids separator of FIG. 1 is in a starting position in the sample container;

FIG. 9 illustrates the solids separator according to FIG. 1 showing different movement stages of the solids separator;

FIG. 10 illustrates a detail view of the solids separator during a centrifugation process;

FIG. 11 illustrates the sample container with the solids separator according to FIG. 1 in a final condition of the solids separator after the centrifugation process;

FIG. 12 illustrates the ballast weight of the solids separator according to FIG. 1 during fabrication.

FIG. 13 illustrates the solids separator of FIG. 1 during a fabrication process;

FIG. 14 illustrates another view of the solids separator of FIG. 1 during a fabrication process;

FIG. 15 illustrates another embodiment of the solids separator according to the invention during a fabrication process.

### DETAILED DESCRIPTION OF THE INVENTION

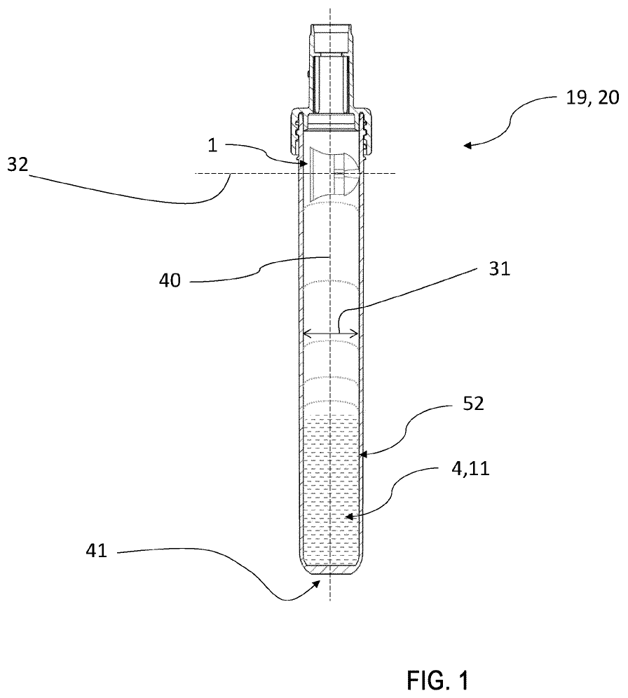

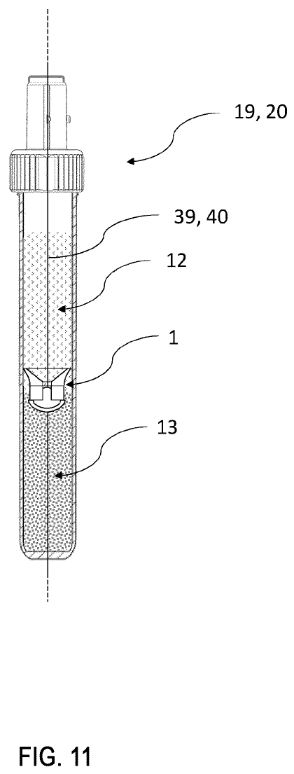

The solids separator 1 according to the invention is illustrated in FIGS. 1-15. The solids separator 1 is used to separate components 2, 3 of a bodily fluid 4 with different densities during a centrifugation process. The centrifugation process is an essential part of pre-analysis of blood 11 for medical purposes. Thus, blood 11 is drawn from a patient and filled into a sample container 19, configured as a blood vial. As evident from FIG. 1, the solids separator 1 is already arranged in the blood vial since the solids separator 1 had previously been inserted into the blood vial. The centrifugation process separates the blood 11 into components 2, 3 namely, blood plasma 12, blood serum 13, and a leukocyte film also designated as buffy coat, wherein the components 2, 3 of the blood 11 have different densities.

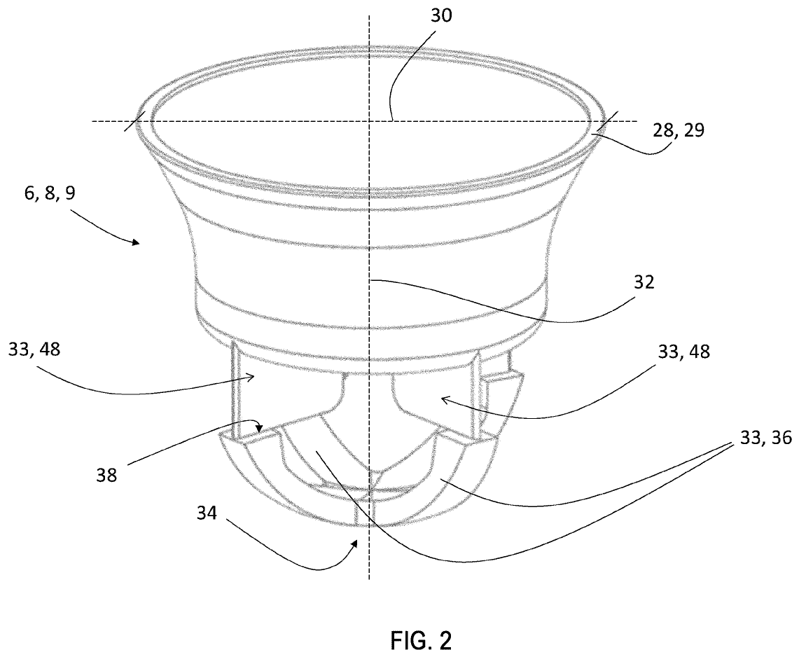

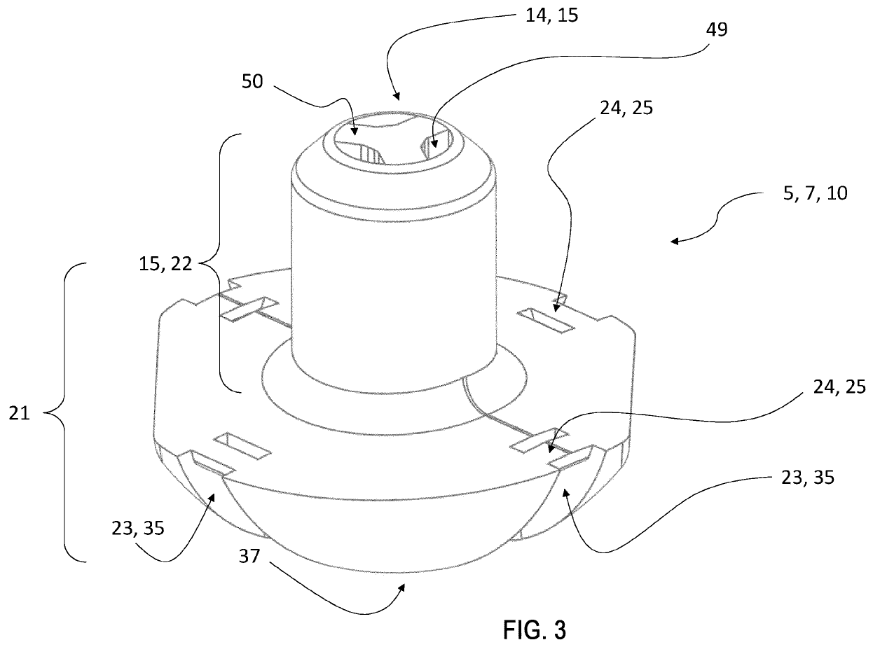

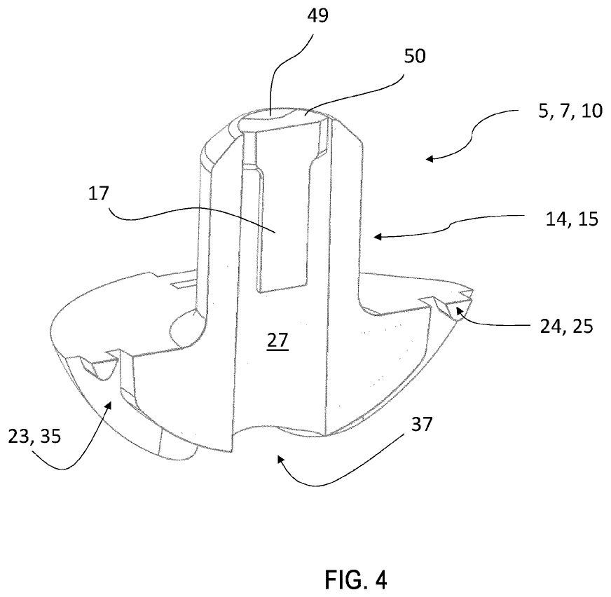

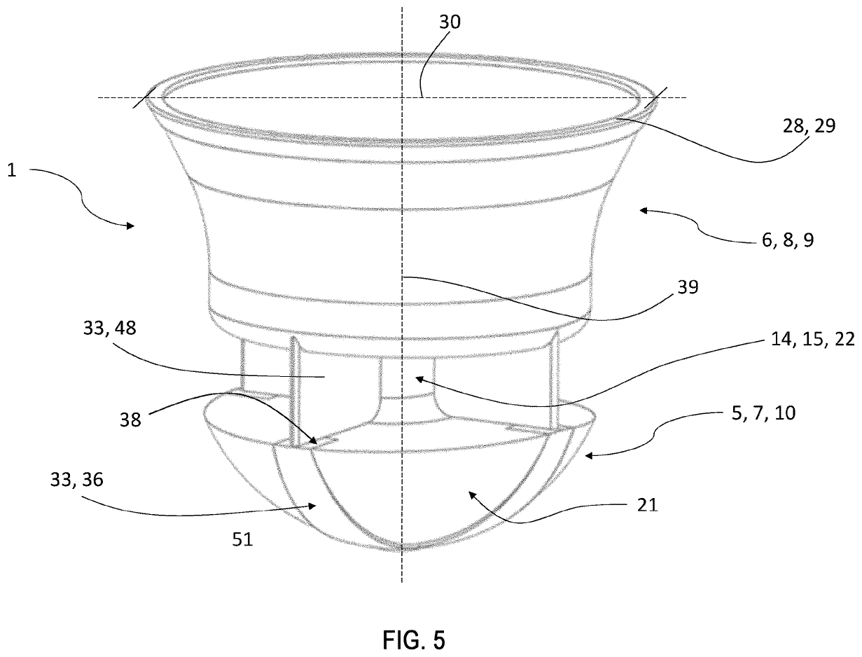

The solids separator 1 includes two components 7, 8 connected with one another and moveable relative to each other, a float 9 and a ballast weight 10. The float 9 is shown in in FIG. 2. The ballast weight 10 is shown in FIGS. 3 and 4. FIGS. 5-7 show an assembled condition of the solids separator 1. The ballast weight 10 is made from a first material 5, whereas the float 9 is made from a second material 6. Both components 7, 8 are made from synthetic materials. The first material 5 forming the ballast weight 10 has a first density ρ₁ that is greater than a second density ρ₂ of the second material forming the float 9. The second density ρ₂ is smaller than a density of the blood serum 13, the first density ρ₁ is greater than a density of the blood plasma 12. An entire density ρₜₒₜₐₗ of the solids separator is between the density of the blood plasma 12 and the density of the blood serum 13.

The second material 6 is additionally configured soft and pliable and therefore has reset properties. The first material 5, however, does not have reset properties and does not have elastic properties.

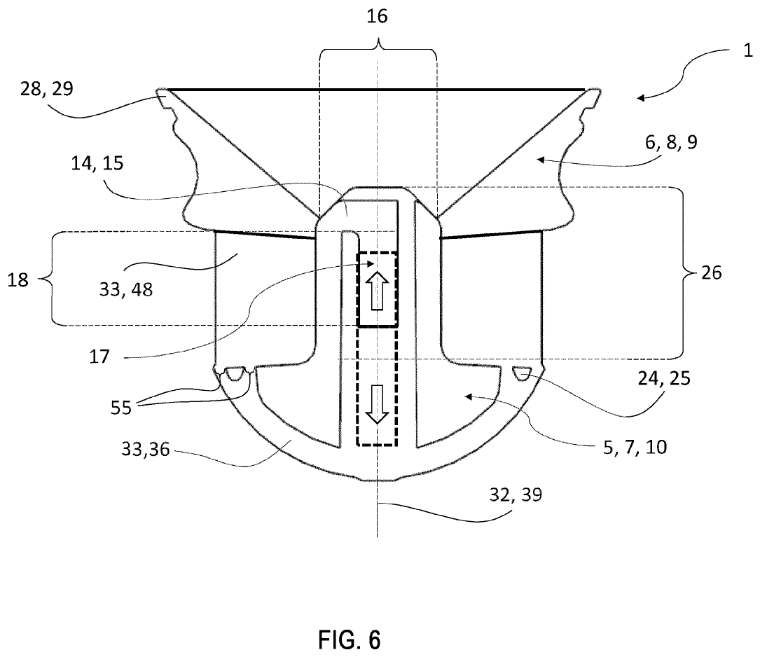

The ballast weight 10 includes a first section 21 that is essentially semi-spherical and second section 22, wherein the second section 22 is configured as a trunnion. The first section 21 of the ballast weight 10 includes four grooves 23 spaced at an angle of 90 degrees from one another. A top side of the first section 21 includes four connection elements 24 configured as bars 25. The bars 25 connect the portions of the first section 21 of the ballast weight 10 that do not include a groove. The trunnion 15 of the ballast weight 10 is configured hollow as evident from FIG. 4 and includes an inner trunnion 17 protruding into a cavity 27. Thus, a length 18 of the inner trunnion 17 is reduced compared to a length 26 of the cavity 27 of the trunnion 15 as evident from FIG. 6.

The float 9 is configured funnel-shaped and includes a sealing portion 28 at an upper end. The sealing portion 28 includes a circumferential sealing edge 29, configured to circumferentially contact and seal and inside of the blood vial 20. An outer diameter 30 of the sealing edge 29 is thus configured larger than an inner diameter 31 of the blood vial 20, so that the solids separator 1 is supported in the blood vial 20 due to a static friction of the sealing edge 29 at the wall 52 when the solids separator is in its idle position illustrated in particular in FIG. 1. The solids separator 9 includes a pass-through opening 16 that extends through the float 9 along a longitudinal axis 32 of the float 9.

The pass-through opening 16 of the float 9 and the trunnion 15 of the ballast weight 10 jointly form a valve 14 for opening or closing the pass-through opening 16 in the float 9. The trunnion 15 of the ballast weight 10 is inserted into the pass-through opening 16 of the float 9 in an idle condition of the solids separator 1 which is illustrated in FIG. 5 and the trunnion 15 closes the pass-through opening 16 liquid tight.

The float 9 and the ballast weight 10, however, are movable relative to each other. A centrifugal acceleration of the solids separator 1 imparts a greater force upon the ballast weight 10 than upon the float 9 so that the ballast weight 10 moves relative to the float 9. Thus, the trunnion 15 is moved out of the pass-through opening 16. The float 9 of the solids separator 1 includes a connection element 33 in order to prevent both components 7, 8 from separating from each other completely and in order to assure that the valve 14 closes again self-acting when the centrifugal force is removed. The connection element 33 thus includes four lobe-shaped sections 48 that are made from the second material 6 and that are respectively arranged at an angle of 90 degrees relative to each other at a bottom side 29 of the float 9. The lobe-shaped sections 48 are elastically deformable and establish a force transferring connection between the float 9 and the ballast weight 10, so that a force is required to overcome a reset force that is imparted by the connection element 33 during a relative movement between the float 9 and the ballast weight 10, starting from an idle position of the solids separator 1. This force is provided by an impact of the centrifugal acceleration upon the solids separator 1.

Additionally, the connection element 33 overlaps the ballast weight 10 in an overlap portion 35. The solids separator 1 includes grooves 23 in the overlap portion 35 in the first section 21 of the ballast weight 10. Additionally, the connection element 33 includes four support bars 36 that envelop the ballast weight 10. The grooves 23 are thus configured to receive the support bars 36. The support bars 36 converge at a bottom side 51 of the solids separator 1. The support bars 36 are made from the second material 6 as well.

Thus, the connection element 33 is connected with the ballast weight 10 in four anchoring locations 38 using four anchoring elements 24, so that it is prevented during a relative movement, in particular, caused by an impact of a centrifugal force upon the solids separator 1, that the respective support bar of the connection element 33 deforms in the overlapping portion 35. The bars 25 in the ballast weight 10 function as anchoring elements 24. The bars 25 are connected on one side with the support bars 36 by positive form locking and connected on the other side with the lobe-shaped sections 48 of the connection element 33 outside of the overlap portion 35.

The float 9 and the connection element 33 are integrally made in one piece from the same material. The lobe-shaped sections 48 of the connection element 33 thus integrally connect the support bars 36 in one piece with the sealing portion 28 of the solids separator 1 as evident from FIG. 2. Thus, it is evident that a cross-sectional surface of the support bars 36 exceeds a cross-sectional surface of the lobe-shaped sections 48 of the connection element 33. Put differently, the support bars 36 are thicker than the lobe-shaped sections 48 of the connection element 33. Both portions of the connection element 33, this means the support bars 36 and lobe-shaped sections 48, are thus connected with one another in a contact area 55. The contact area 55 in an area of the anchoring locations 38 and the support bars 36 and the lobe-shaped sections 48, however, is configured small as evident from FIG. 6. The contact area 55 can be divided into two sections which are respectively adjacent to the respective bar 36. The support bars 36 and the lobe-shaped sections 48 thus function mechanically as two springs with different spring constants that are connected in series. Thus, a spring constant associated with the support bars 36 is greater than a spring constant associated with the lobe-shaped sections 48, which causes only the lobe-shaped sections 48 to deform under force impact but does not cause the support bars 36 to deform.

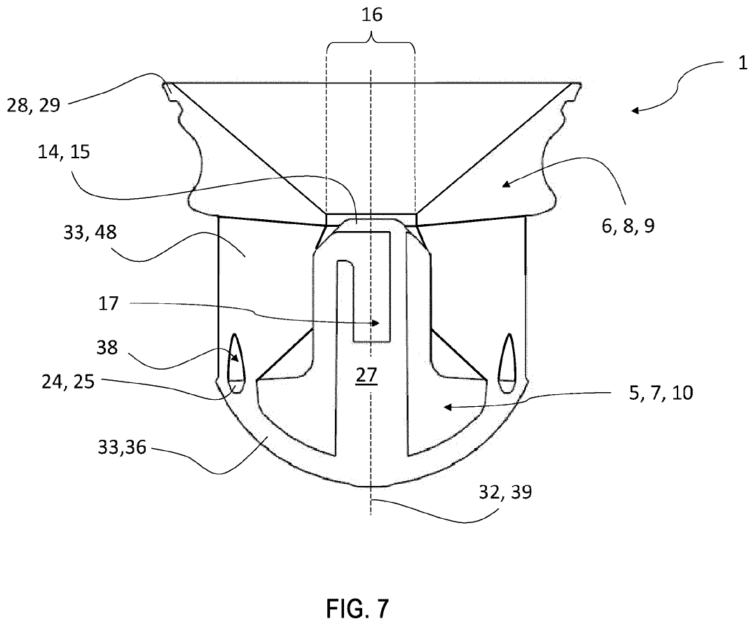

The solids separator 1 that is impacted by a centrifugal force is shown in FIG. 7. The centrifugal force caused by the centrifugation process causes a separation of the ballast weight 10 from the float 9. The connection element 33 therefore causes the ballast weight 10 to not completely separate from the float 9. Thus, the connection element 33 is elastically deformed in an area outside of the overlap portion 35, namely, in the area of the lobe-shaped section 48. The support bars 36, however, are not being deformed due to the connection of the connection element 33 through the anchoring elements 24, the small contact area 55 with the lobe-shaped sections 48 and the associated spring constant.

Simultaneously, the ballast weight 10 is moved away from the float 9, so that a contact between the lobe-shaped sections 48 of the connection element 33 and the first section 21 of the ballast weight 10 outside of the anchoring location 38 is released during a duration of the centrifugation process as evident, in particular, from FIG. 7. The contact of the lobe-shaped sections 48 of the connection element 33 and the anchoring elements 48 is at least released partially on one side. However, the lobe-shaped sections 48 of the connection element 33 and the support bars 36 of the connection element 33 remain in contact even under the impact of the centrifugal force due to the one piece integral connection between the lobe-shaped sections 48 of the connection element 33 and the support bars 36 of the connection element 33.

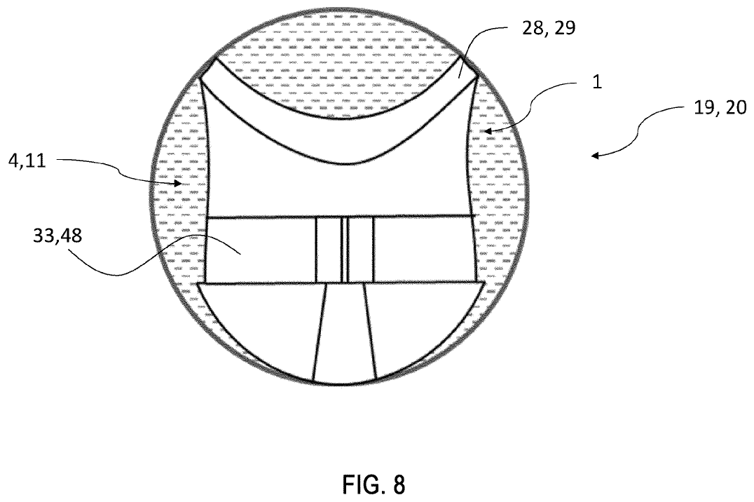

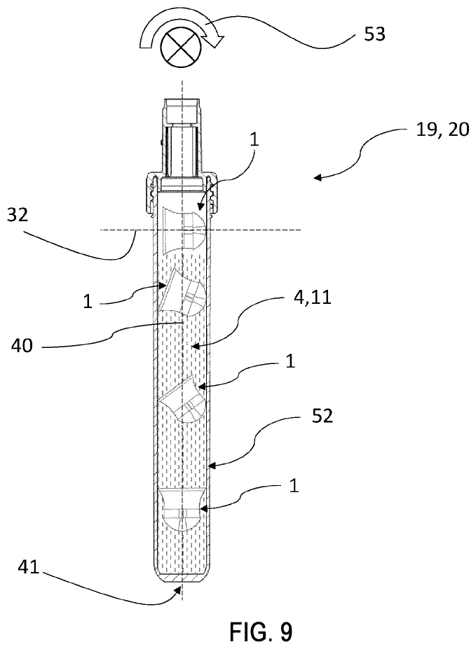

The sealing portion 28 of the float 9 is compressed when inserted into the blood vial 20, so that the solids separator 1 can be arranged in the blood vial 20. Thus, the solids separator 1 is rotated by 90 degrees with its longitudinal axis 39 relative to an idle position that is shown in FIGS. 2-7, so that the longitudinal axis 39 of the solids separator 1 is arranged perpendicular to a longitudinal axis of the blood vial 20. The inserted position of the solids separator 1 is shown in FIG. 8. The blood 11 of the patient can be filled into the blood vial 20 after insertion of the solids separator 1. Thereafter, the blood vial 20 with the solids separator 1 is inserted into a centrifuge and centrifuged. A rotation direction 53 in which the blood vial 20 is rotated is thus illustrated in FIG. 9.

The solids separator 1 overcomes the static friction due to the centrifugal force impacting the solids separator 1. The solids separator 1 moves towards a base 41 of the blood vial 20 and pivots, since the centrifugal force has a greater impact upon the ballast weight 10 due to its density being greater than the density of the float 9. The solids separator 1 thus aligns so that the longitudinal axis 39 of the solids separator 1 is oriented parallel to the longitudinal axis 40 of the blood vial 20. The sealing edge 29 contacts an inside of the blood vial 20 in a sealing manner in this condition.

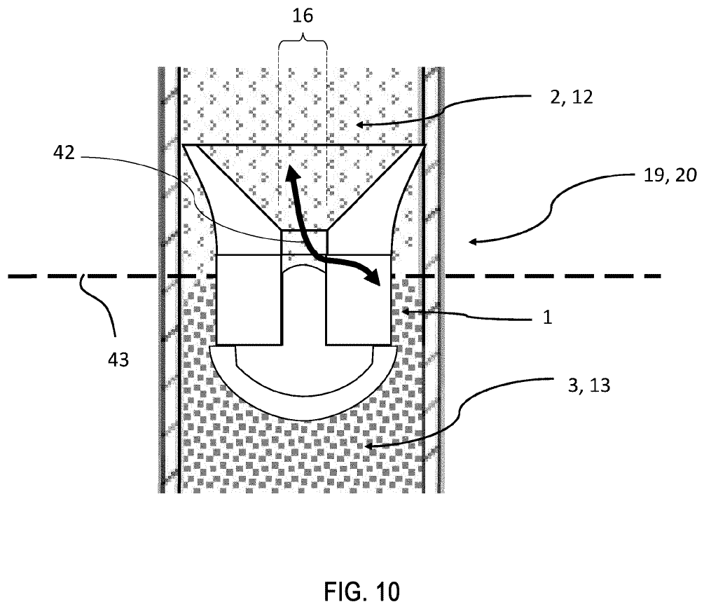

The components 2, 3 of the bodily liquid 4 that is to be separated are simultaneously separated from each other during the centrifugation. The separation of the components 2, 3 is facilitated by the presence of the pass-through opening 16 in the float 9. The trunnion 15 of the ballast weight 10 is moved out of the pass-through opening 16 during centrifugation, so that a flow path 42 for the components 2, 3 of the blood 11 is released as evident from FIG. 10. This way, the components 2, 3 arrange themselves above and below the solids separator 1 as a function of their density.

Since the total density ρₜₒₜₐₗ of the solids separator 1 is between the density of the first component 2 and the density of the second component 3 and the density of the bodily liquid 4, the solids separator 1 moves to a boundary 43 between the two components 2, 3 after separation of the two components 2, 3 as evident from FIG. 10. Thus, the total density ρ_{Total} of the solids separator 1 is advantageously selected so that a lowest spot of the pass-through opening 16 of the solids separator 1 is arranged above the boundary 43 between the two components 2, 3. In an exemplary manner, the distance between the lowest spot and the boundary 43 may be approximately 1 mm. This way, a safety distance can be selected that prevents that the second component 3 can move above the solids separator 1 after the centrifugal force ceases. By the same token, the distance is selected as small as possible so that a loss of sample material, in particular of the component 2, is minimized after pouring the component 2 out of the blood vial 20 after completion of the centrifugation process.

FIG. 9 shows different movement phases of the solids separator 1. The connection element 33 causes the valve 14 to close after the centrifugal force ceases when the centrifugation process is completed. Thus, the trunnion 15 of the ballast weight 10 is inserted back into the pass-through opening 16. This way, the components 2, 3 of the bodily fluid 4 remain separated from each other even when the centrifugal force cease.

The reset force of the lobe-shaped sections 48 of the connection element 33 is thus selected so that the trunnion 15 of the ballast weight 10 is already inserted back into the pass-through opening 19 when the centrifugal force is reduced and does not move back into the pass-through opening 16 when the centrifugal force has ceased completely. This condition is shown in FIG. 7. Fabrication of the solids separator 1 assures that the solids separator 1 moves to the boundary 43 of the components 2, 3, even when there are variations in the thicknesses ρ₁, ρ₂ of the two materials that are being used.

The total density ρₜₒₜₐₗ of the solids separator 1 is computed as follows:

$${ρ}_{total} = \frac{m 1 + m 2}{V 1 + V 2} = \frac{ρ 1 \cdot V 1 + ρ 2 \cdot V 2}{V 1 + V 2}$$

Variations in the thickness ρ₁ of the first material 5 and/or the thickness ρ₂ of the second material 6 can cause a variation of a value of the total thickness ρ_{Total} of the solids separator 1 from a nominal value of the total thickness ρ_{Total} of the solids separator 1 wherein the nominal value is required for an optimum separation of the components 2, 3 of the blood 11. In order to prevent this, the densities ρ₁ and ρ₂ of the two materials 5, 6 are determined in a first step and compared with the respective nominal densities. The nominal densities are the densities that the materials 5, 6 have to have so that the value of the total density ρₜₒₜₐₗ of the solids separator 1 is adjusted in an optimum manner.

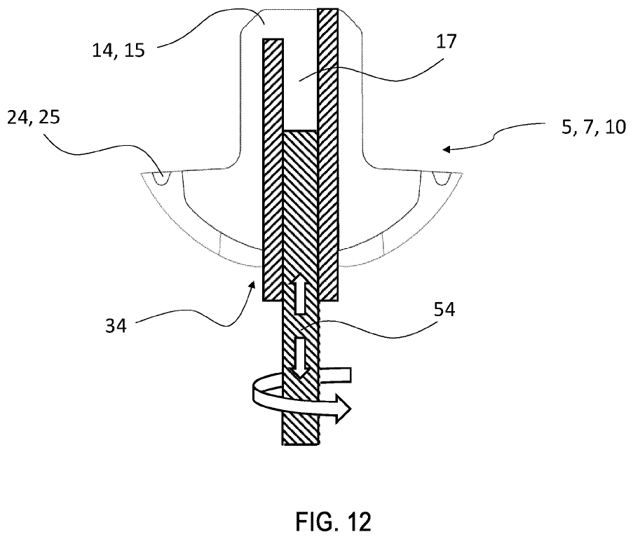

A volume V₁, V₂ of the first material 5 and/or of the second material 6 is adjusted as a function of the material 5, 6 whose thickness ρ₁, ρ₂ deviates from the nominal thickness. The adjustment of the volume V₁, V₂, is thus performed e.g. by an extension or shortening of the inner trunnion 17 of the trunnion 15 of the ballast weight 10 of the solids separator 1 as illustrated in FIG. 12.

When the thickness ρ₁ of the first material 5 that forms the ballast weight 10 is smaller than the nominal thickness, the value of the total thickness ρ_{Total} can be adjusted by extending the inner trunnion 17. Vice versa, the value of the total thickness ρ_{Total} can be adjusted by shortening the inner trunnion 17 when the thickness ρ₁ of the first material 5 is greater than the nominal thickness. The volume V₁ of the first material 5 that is used to form the solids separator 1 can thus be determined as follows.

$${V}_{1} = \frac{{V}_{2} ( {ρ}_{Total} - {ρ}_{2} )}{{ρ}_{2} - {ρ}_{Total}}$$

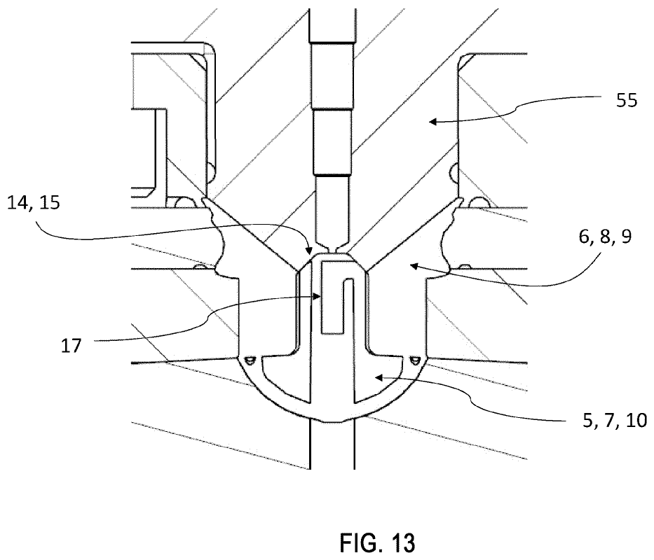

The solids separator 1 is thus produced by injection molding as illustrated in FIG. 13. Thus, the first material 5 is injected into a first forming tool 55 in a first method step, which forms a first component 7. The first component 7 subsequently forms the ballast weight 10 of the solids separator 1. The inner trunnion 17 and a wall of the trunnion 15 do not contact so that a cavity 44 is formed in the trunnion 17. A top side of the trunnion 15 includes an annular recess 49 as evident from FIG. 4, wherein the recess 49 is closed at three connection locations between the inner trunnion 17 and the trunnion 15.

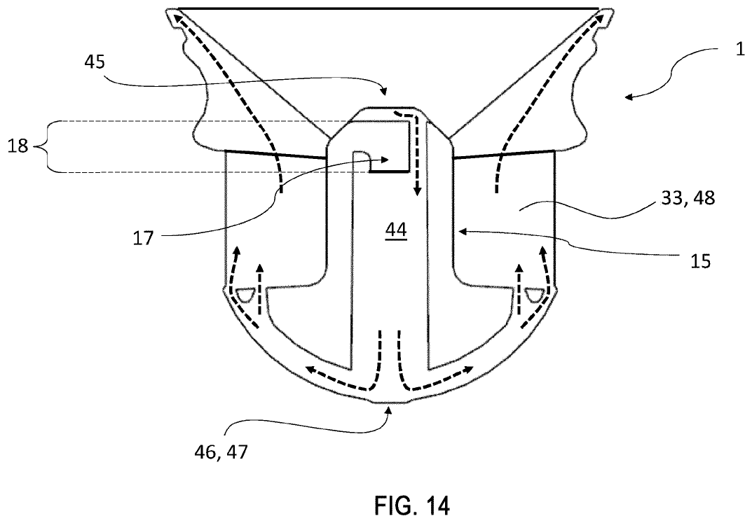

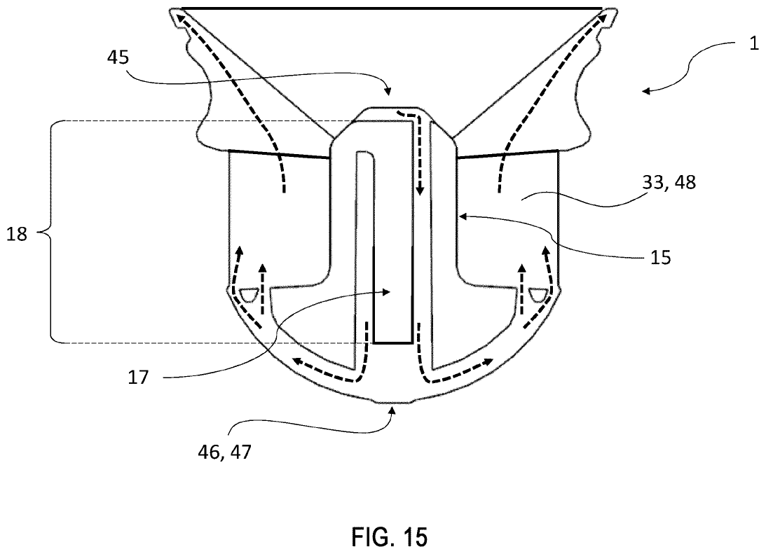

After the first material 5 is cured, the forming tool 55 is moved from a first forming position into a second forming position. A cavity enclosed by the forming tool 55 in the second forming position thus essentially corresponds to the final shape of the solids separator 1. The second material 6 is eventually injected into the forming tool 55 in a second method step through the annular recess 49 of the ballast weight 10 without forming a bonded connection with the first material 5 or the first component 7 formed therefrom. Thus, the second material 6 flows from the annular recess 49 at the top side 45 of the first component 7 through the cavity 44 in the trunnion 15 to a bottom side 46 of the first component 7 as evident from FIGS. 14 and 15. Thus, the bottom side 46 forms a reversal area 47, wherein the second material 6 flows through the reversal area about an outside of the first component 7 to a top side 45 of the first component 7. The second material 6 fills the cavity 44 in the trunnion 15 of the first component 7 completely in a final condition. The second material 6 is eventually cured as well. The two components 7, 8 are moveable relative to each other after curing the second material 6, which prevents a complete separation of the float 9 from the ballast weight 10 due to the friction locked connection of the connection element 33 in the anchoring locations 38.

A second tool 54 can be used to adjust a length 18 of the inner trunnion 17 during the fabrication process and thus adjust a volume of the first component 7, wherein the additional tool 54 is used before the material 5 is injected into the forming tool 55 as evident in particular from FIG. 12. Thus, the additional tool 54 defines a flow of the first material 5 in a direction towards a bottom side 34 of the float 9. This facilitates adjusting a length 18 of the inner trunnion 17. The additional tool 54 can be removed after curing and inserting the first component 7 into the second forming tool 55 or after transferring the second forming tool 55 into the second forming position.

The solids separator 1 thus produced can have inner trunnions 17 with different lengths as evident from FIGS. 14 and 15 while the total density ρ_{Total} remains constant. FIG. 14 shows a solids separator 1 including a short inner trunnion 17 while the solids separator 1 illustrated in FIG. 15 includes a much longer inner trunnion 17, wherein a length 18 of the inner trunnion 17 exceeds a length of the trunnion 15 and penetrates the first section 21 of the ballast weight 10.

### REFERENCE NUMERALS AND DESIGNATIONS

1 solids separator
2 first component
3 second component
4 bodily fluid
5 first material
6 second material
7 first component
8 second component
9 float
10 ballast weight
11 blood
12 blood plasma
13 blood serum
14 valve
15 trunnion
16 pass-through opening
17 inner trunnion
18 length of inner trunnion
19 sample container
20 blood vial
21 first section
22 second section
23 groove
24 anchoring element
25 bar
26 length of trunnion
27 cavity
28 sealing portion
29 sealing edge
30 outer diameter of sealing edge
31 inner diameter of blood vial
32 longitudinal axis of float
33 connection element
34 bottom side of float
35 overlap portion
36 support bar
37 bottom side of ballast weight
38 anchoring location
39 longitudinal axis of solids separator
40 longitudinal axis of blood vial
41 base of blood vial
42 flow path
43 boundary
44 cavity
45 top side of first component
46 bottom side of first component
47 reversal area
48 section
49 recess
50 connection location
51 bottom side of solids separator
52 wall
53 rotation direction
54 tool
55 forming tool
56 contact area
ρ₁ first density
ρ₂ second density
ρ_{Total} total density
V₁ first volume
V₂ second volume

What is claimed is:

1. A solids separator, comprising:
an axis;
a ballast weight;
a float,
wherein the solids separator is configured to separate two components of a bodily fluid, wherein a first component of the bodily fluid has a first density and a second component of the body fluid has a second density, and wherein a total density of the solids separator is between the first density and the second density of the two components of the bodily fluid that is to be separated,
wherein the ballast weight and the float are movable relative to each other under an influence of a centrifugal force acting upon the solids separator;
wherein the solids separator includes an elastically deformable connection element forming a force transmitting connection between the float and the ballast weight, so that a relative movement between the float and the ballast weight from an idle position requires a force to overcome a reset force caused by the connection element,
wherein the connection element axially overlaps the ballast weight or the float in an overlap portion,
wherein the connection element is connected with the ballast weight or the float in an anchoring location, so that a deformation of the connection element in a section of the overlap portion is prevented during the relative movement between the ballast weight and the float caused by a centrifugal force impacting the solids separator floating in the bodily fluid.

2. The solids separator according to claim 1, wherein the ballast weight is connected with the float by friction locking or positive form locking.

3. The solids separator according to claim 1, wherein the connection element includes at least one lobe shaped section.

4. The solids separator according to claim 1, wherein the connection element is essentially formed from an identical material as the float.

5. The solids separator according to claim 1,
wherein the connection element is connected with the ballast weight or the float by an anchoring element through positive form locking, and
wherein the anchoring element is configured as a bar or a trunnion.

6. The solids separator according to claim 1, wherein the float and/or the ballast weight includes at least one groove in an axial overlap portion.

7. The solids separator according to claim 1, wherein the connection element includes at least one support bar that at least partially envelops the float or the ballast weight.

8. The solids separator according to claim 7, comprising a plurality of support bars.

9. The solids separator according to claim 8,
wherein the connection element includes four lobe-shaped sections and four support bars associated with the lobe-shaped sections, and
wherein the lobe-shaped sections and the associated support bars are respectively arranged at an angle of 90 degrees from one another.

10. The solids separator according to claim 8, wherein the support bars converge in an end portion of the solids separator.

11. The solids separator according to claim 7,
wherein the float includes a sealing portion, and
wherein the sealing portion is bonded to the support bar by the connection element.

12. The solids separator according to claim 7, wherein a cross-sectional surface of the support bars exceeds a cross-sectional surface of the lobe-shaped sections of the connection element.

13. The solids separator according to claim 7,
wherein the ballast weight includes a first section and a second section, and
wherein the first section is configured semi-spherical and the second section is configured trunnion-shaped.

14. The solids separator according to claim 13,
wherein the float includes a pass-through opening, and
wherein the second section of the ballast weight is insertable into the pass-through opening to form a valve.

15. The solids separator according to claim 13, wherein the first section of the ballast weight includes grooves at least partially enveloping the first section.

16. The solids separator according to claim 15, wherein the support bar runs in the grooves of the ballast weight.

17. The solids separator according to claim 13, wherein the connection element is deformable during a relative movement between the float and the ballast weight caused by the centrifugal force impacting the solids separator, so that a contact between the connection element and the first section of the ballast weight outside of the anchoring location is temporarily disengageable at least partially.

18. The solids separator according to claim 1, wherein the first density is greater than 1.05 g/cm³, the second density is less than 1.03 g/cm³, and the total density is in a range of 1.03 g/cm³ to 1.05 g/cm³.
